(19) United States
(12) Patent Application Publication
(10) Pub. No.: US 2020/0210804 A1
(43) Pub. Date: Jul. 2, 2020

(54) INTELLIGENT ENCLOSURE SYSTEMS AND COMPUTING METHODS

(71) Applicant: Qi Lu, Bellevue, WA (US)

(72) Inventor: Qi Lu, Bellevue, WA (US)

(21) Appl. No.: 16/571,315

(22) Filed: Sep. 16, 2019

Related U.S. Application Data
(60) Provisional application No. 62/786,600, filed on Dec. 31, 2018.

(51) Int. Cl. G06N 3/00; G06N 3/08; G16H 50/20; G05B 19/042

(52) U.S. Cl. CPC G06N 3/006; G06N 3/08; G05B 2219/23238; G05B 19/042; G16H 50/20

(57) ABSTRACT

A system comprising a physical sphere, a digital sphere and a fusion system. The physical sphere including physical spatial elements and temporal elements. The fusion system comprising a foreplane including physical fabric, a perceptor subsystem, and an actuator subsystem, and a backplane including a communication infrastructure, computing and storage infrastructure, power infrastructure, redundancy, and cloud connections. The digital sphere including an artificial intelligence system tethered to the physical sphere, the artificial intelligence system comprising a subsystem of observation configured to receive data from the perceptor subsystem, a subsystem of thinking configured to learn from, model, and determine a state of an enclosure based on the received data, and a subsystem of activity configured to generate decisions with the actuator subsystem based on the state of the enclosure according to a predetermined objective for the enclosure. Computations performed by the system are spatial tethered where operations require spatial signatures to ensure information is contained within the enclosure.

### CROSS REFERENCE TO RELATED APPLICATION

This application claims the priority of U.S. Provisional Application No. 62/786,600, entitled “INTELLIGENT ENCLOSURE SYSTEMS AND COMPUTING METHODS,” filed on Dec. 31, 2018, the disclosure of which is hereby incorporated by reference in its entirety.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE INVENTION

### Field of the Invention

This application generally relates to artificial intelligence and societal infrastructures that are enabled by it, and in particular, a system platform architecture for creating environments driven by artificial intelligence.

### Description of the Related Art

Artificial intelligence (“AI”) is an example of one of the most general and most potent general-purpose technologies ever invented. Past computing systems have been primarily for human-digital computing interaction, but AI has the capacity to automatically and efficiently acquire knowledge and apply the knowledge to achieve goals. AI technologies such as deep learning enable efficient and rapid distillation of knowledge from data. AI systems may become integral to everyday human life by interacting with physical and biological worlds.

There's a need to apply AI technologies to the future of work and life environments through the development and deployment of AI-powered infrastructures.

### SUMMARY OF THE INVENTION

The present invention provides a system, apparatus, and methods for converting physical enclosures or enclosed spaces into intelligent computing systems. According to one embodiment, the system may comprise a physical sphere, a digital sphere and a fusion system. The physical sphere may include physical spatial elements and temporal elements. The digital sphere may include an artificial intelligence (“AI”) system coupled to the physical sphere by a fusion system. The AI system comprises a subsystem of observation configured to receive data from the perceptor subsystem, a subsystem of thinking configured to learn from and model the received data, and a subsystem of activity configured to generate decisions with actuators based on the learning and modeling of the subsystem of thinking. The fusion system may comprise a foreplane including physical fabric, a perceptor subsystem, an actuator subsystem, and an administrator console, and a backplane including a communication infrastructure, computing and storage infrastructure, power infrastructure, redundancy, and cloud connections.

According to another embodiment, the system may comprise a physical sphere including physical spatial elements and temporal elements associated with an enclosure, a fusion system comprising a foreplane including physical fabric, a perceptor subsystem, and an actuator subsystem, and a backplane including a communication infrastructure, computing and storage infrastructure, power infrastructure, redundancy, and cloud connections, and a digital sphere including an artificial intelligence (“AI”) system coupled to the physical sphere by the fusion system. The AI system may comprise a subsystem of observation configured to receive data from the perceptor subsystem, the data corresponding to the physical spatial elements and the temporal elements, a subsystem of thinking configured to learn from, model, and determine a state of the enclosure based on the received data, and a subsystem of activity configured to generate decisions with the actuator subsystem based on the state of the enclosure according to a predetermined objective for the enclosure.

A perceptor subsystem may comprise one or more devices that include one or more sensors, on-sensor computing silicon, and embedded software. In one embodiment, the perceptor subsystem may comprise at least one of optical, auditory, motion, heat, humidity, and smell sensors. In another embodiment, the perceptor subsystem may comprise at least one of phone, camera, robotic, drones, and haptic devices. In yet another embodiment, the perceptor subsystem may comprise medical equipment that assesses a state of health for biological actors within the enclosure.

The enclosure may comprise an enclosed physical space that serves a defined social economical purpose. The subsystem of thinking may be configured to model the received data according to a domain theme. A given enclosure may have its associated social/societal and/or natural meaning and related thematic focus based on the domain theme. For example, the domain theme may include at least one of a retail floor, school, hospital, legal office, trading floor, and hotel. In one embodiment, the generated decisions include tasks to achieve functions according to the domain theme. The subsystem of thinking may be further configured to build a model of the physical sphere, wherein the model includes a description of a semantic space and ongoing actions of the physical sphere. The AI system may be configured to train the model by learning relationships and responses to satisfy given goals or objectives based on a domain theme. The AI system may be further configured to calibrate the learned relationships based on configurations including at least one of settings, preferences, policies, rules, and laws. The subsystem of thinking may be further configured to use domain-specific deep-learning algorithms and overall life-long learning to improve the model.

The state of the enclosure may comprise a combination of the physical spatial elements and the temporal elements that is monitored by the AI system. The backplane may be spatial-aware and the communication infrastructure, computing and storage infrastructure, power infrastructure, redundancy, and cloud connections of the backplane may be tagged with spatial-signatures that prohibit tampering. The backplane can perform computation operations that ensure information is contained within the physical enclosure. The physical spatial elements may comprise features associated with a geometry of the enclosure including separating structures, an interior and exterior of the enclosure, objects, actors, and environment. The temporal elements may include factors related to time, events, and environmental changes. The subsystem of activity may be further configured to use the actuator subsystem to induce changes in the physical sphere based on the generated decisions. The actuator subsystem may comprise digital controls for equipment, appliance, mechanical, and perimeter objects.

### BRIEF DESCRIPTION OF THE DRAWINGS

The invention is illustrated in the figures of the accompanying drawings which are meant to be exemplary and not limiting, in which like references are intended to refer to like or corresponding parts.

FIG. 1 illustrates an intelligent enclosure system according to an embodiment of the present invention.

FIG. 2 illustrates a computing and storage infrastructure according to an embodiment of the present invention.

FIG. 3 illustrates an artificial intelligence system according to an embodiment of the present invention.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. It is to be understood that other embodiments may be utilized and structural changes may be made without departing from the scope of the present invention. Likewise, a reasonably broad scope for claimed or covered subject matter is intended. Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of exemplary embodiments in whole or in part. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

In general, the present systems and methods disclosed herein provide for environments where a physical sphere including perceptors, actuators, and other devices powered by digital computing and artificial intelligence (“AI”) may be fused together or inseparably integrated with space and time of the physical world within an enclosure space. The environments may be configured with rules and actions across a plurality of devices to control such devices concurrently, and/or have such devices operate automatically, for instance, according to desired spatial settings, experiences, or goals. The environments may accommodate and assimilate spatial form factors that account for geometry of an enclosed space via separators (e.g., wall, floor, ceiling, open-space perimeter), functional components (e.g., door, window, etc.), interior and exterior (shape, color, material: wood, brick, etc.), objects (physical entities contained within (furniture, appliance) and adjacent of the exterior), actors (e.g., biological (human, animal) or mechanical (robots, drones)), and environment (e.g., temperature, air, lighting, acoustic, utility (power, water) etc.). Temporal dimension factors including present, past, history of events, activity sequence of actors, and environment changes may also be assimilated by the environment.

Spatial and temporal factors may be recognized and tracked using, for example, optical sensors (e.g. camera, depth camera, time-of-flight (“TOF”) camera, etc.) and computer vision algorithms based on deep-learning. Other aspects, such as actor motion, can be recognized and tracked via motion sensors. Physical environment factors such as temperature can be tracked via thermal sensors. The storing and capture of these factors can be performed using comprehensive model training and long-term and life-long learning systems that may capture the semantics of the physical factors, as well as domain specific models about an enclosure, e.g., the semantics of people wearing white gown may be a doctor in an enclosure that is a hospital.

The disclosed systems may also include a digital sphere comprising a subsystem of observation, a subsystem of thinking and a subsystem of thinking. The subsystem of observation may be configured to use perceptors to observe an environment associated with the system. A perceptor may include a corresponding sensor and an on-sensor computing module to process analog signals, e.g., from the environment, and organize them into digital representations. Examples of perceptors may include a TOF camera-array with on-sensor silicon and native neural-networks, and a microphone array with silicon consists of DSP and neural-network.

The subsystem of thinking may be configured to generalize and memorize the data from the subsystem of observation. The subsystem of thinking may include a set of “learner” or “modeler” computing elements that build/maintain models of the environment. The models may describe a semantic space and ongoing actions (e.g., a state of the enclosure) for a physical enclosure associated with the system. The subsystem of thinking may be configured to use deep learning as a basic computing substrate, while applying a variety of domain-specific deep-learning algorithms and an overall life-long learning regime to build/refine/improve the models about the environment that the enclosure is intended for, such as a classroom, a hospital operating room, etc.

A subsystem of activity may be configured to use actuators that are physically connected with the environment to carry out certain functions in the physical or biological world. The subsystem of activity may apply controls based on provisioned “objectives” of an overall AI-system. The subsystem of activity can induce changes of the environment through actuators to achieve the “objectives.” Actuators may comprise digital controls for lights, heating/cooling, window shades, and various equipment within the enclosure.

The disclosed systems may be used to provide spatial experiences that can transform and elevate all existing industries (e.g., manufacture, financial service, health care, education, retail, etc.) and all lines of work (e.g., lawyers, doctors, analysts, customer service professionals, teachers, etc.). According to one embodiment, an environment comprises an intelligent enclosure architecture for structural settings, such as a school, hospital, store, home, or workplace. The environment may be configured to perform functions and provide experiences specific to the structural settings for actors interfacing with the environment (e.g., teacher, doctors, customers, housewives, workers) in addition to objects, events, and environment. The functions and objectives may be modeled according to needs and objectives for the structural settings. For each enclosure, a full semantic space can be computed and maintained. The semantic space may capture and describe required information and semantic knowledge for a given enclosure, e.g., a classroom. The semantic space can be domain-specific and can be provided when the enclosure is set-up. For example, in the case where an enclosure is a classroom, a semantic ontology of a classroom may be provided. Machine learning (e.g., deep learning) can then be applied to build models that conform with such ontological semantics such that the meaning of the models may be interpreted, e.g., a teacher is telling a story to a group of 12 children, etc., and used to achieve a required objective specific to the enclosure.

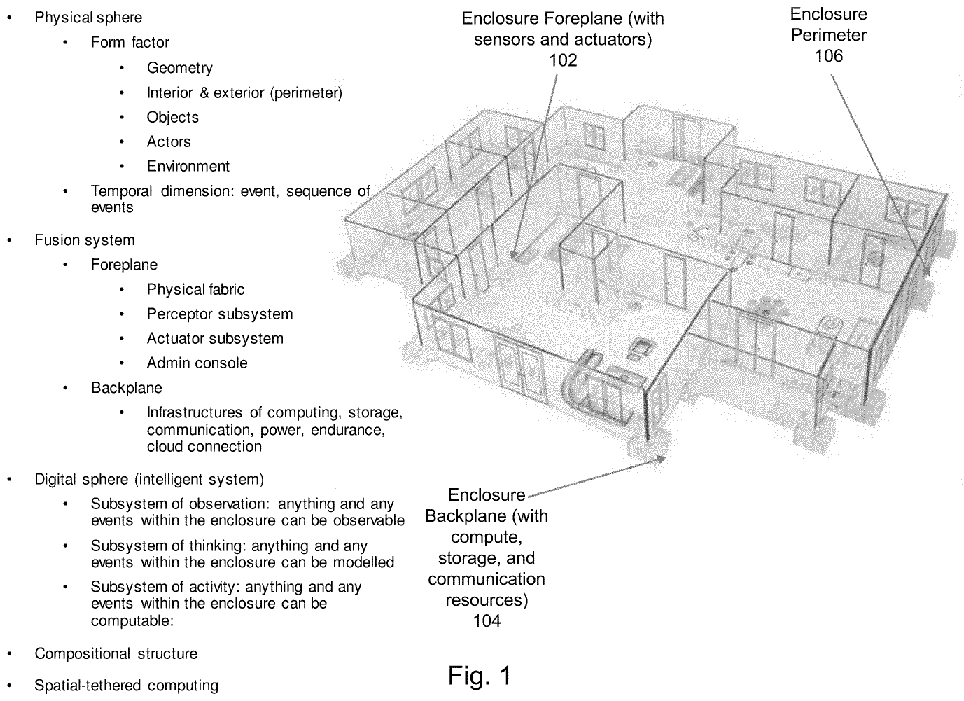

FIG. 1 illustrates an intelligent enclosure system according to an embodiment of the present invention. The intelligent enclosure system presented in FIG. 1 includes a physical sphere, a digital sphere, and a fusion system. The physical sphere may comprise spatial elements related to physical objects of the intelligent enclosure and temporal elements related to time, events, and environmental changes. Examples of spatial elements may include features associated with the geometry of an enclosed space via separators (e.g., walls, floors, ceilings, and open-space perimeters, and functional components, such as door, window, etc.), interior and exterior of the enclosed space (e.g., shape, color, material: wood/brick/etc.), objects (e.g., physical entities that are contained within (furniture, appliance) and adjacent of the exterior), actors (biological (human, animal) or mechanical (robots, drones)), and environment (temperature, air, lighting, acoustic, utility (power, water), etc.). The digital sphere may include an artificial intelligence (“AI”) system that can be fused to the physical sphere by the fusion system. The fusion system may include a foreplane 102, a backplane 104, and an enclosure perimeter 106. The foreplane 102 may comprise physical fabric, a perceptor subsystem, an actuator subsystem, and an administrator console. The physical fabric may include components, such as wires and connected boards/modules that are mounted and integrated within a physical perimeter (wall/floor/ceiling).

The perceptor subsystem enables the projection of the physical sphere of the environment into a digital sphere. The perceptor subsystem may include perceptor sockets that are attached to physical fabric elements. Perceptor sockets may be either in the exterior or interior of an enclosure of the environment, such as the enclosure perimeter 106. The perceptor sockets may comprise (smart) sensors of a variety types, such as optical, auditory, motion, heat, humidity, smell, etc. The perceptor subsystem may also include on-sensor silicon for computation and smart-features (e.g., energy efficiency) and communication fabric (wired and wireless) to the backplane 104 (e.g., for transmission of perceptor data and perceptor control).

The perceptor subsystem may include other types of perceptors, such as non-stationary (phone, camera), wireless or wired (with sockets) connections, mobile perceptors (robots, drones) with wireless connections, and non-remote (haptic) sensors. Special perceptors may also be used to sense actors (human, animal, etc.). For example, the special perceptors may include medical equipment that can measure body temperature and blood pressure, etc., as a way of assessing the state of health for biological actors, such as humans and animals. Perceptors may be localized (relative to the enclosure) and calibrated (perceptor-specific, position, and angle, etc.) that enables spatial awareness and integration with the enclosure. As an example, a perceptor subsystem for a senior citizen home may include optical and motion sensors that are mounted on the wall or placed on the floor. These sensors can detect sufficient data to enable the overall intelligent enclosure to decide whether the senior citizen is trying to get up on the bed in the dark so that the intelligent enclosure can automatically turn on the lights, or if the senior citizen is falling on the ground and not able to get up so that the intelligent enclosure can send an alert to others for further assistance. As another example, optical and motion sensors can be mounted on the wall, roof, placed on shelf, on a retail floor, which can capture data about shopper behavioral data, e.g., how they walk the floor, how they look at different products for different aisles and different product placement on the shelf, etc. This may enable the intelligent enclosure to provide highly useful analytics for the store owners to derive actionable insights as to how to systematically optimize the floor layout and product placement to create better customer experience and increase sales.

The actuator subsystem enables controls and actions with intended goals from the digital sphere to the physical sphere. The actuator subsystem may include wires and boards that are mounted and integrated within the physical perimeter (e.g., floor, wall, ceiling). The actuator subsystem may further include actuator sockets mounted on the interior and the exterior as needed (e.g., embedded within the structure, e.g., wall). Each actuator socket can be plugged with a “control-by-wire” (digital to physical) connector that can interface with any physical controls (light switch, window shades, door lock, air filter, air conditioner, etc.), as well as controllers for enclosed objects such as appliances (e.g., TV remote control). The actuator subsystem may include non-stationary actuator sockets via wireless connection (e.g., universal remote, smart-phone), that wirelessly connected. The actuator subsystem may further include mobile actuators (e.g., robots) via wireless control. Actuator extensions via mechanical and electrical controls may be used for control of objects (furniture/appliances), or the physical perimeters (wall, floor, ceiling, functional modules). An interface for human interaction with the actuator subsystem may be provided to facilitate actions and results to be modeled and enabled within the digital sphere (e.g. via smartphone, or neural-link). The actuator subsystem may also be used to control animals through physical devices and human input. As an example, in the previous case of a senior citizen home, actuators may be placed near the physical switches for the lighting of the rooms so that the lights may be turned on or off automatically. Actuators may also be placed near the physical controls of air-conditioners, ventilators, etc. to maintain the temperature and air quality of the room that suits the senior citizen's health conditions.

The administrator console may comprise a module for controlling configurations of the intelligent enclosure system and providing an outlet (e.g., display) for information, insights, etc.

The backplane 104 comprises on-enclosure computing fabric that includes physical systems that enable the digital sphere of the intelligent enclosure system. The physical systems may include communication infrastructure (wired (with installed/embedded wiring) and wireless (e.g., a WiFi+ on-enclosure base-station), spatial aware data packets (narrowband Internet-of-things-like)), computing and storage infrastructure (e.g., computers or servers), power infrastructure (e.g., power feed from outside of the enclosure, on-enclosure renewable sources or stored sources), on-enclosure digital durability/redundancy (storage redundancy, power supply redundancy), and connections to public and/or private (hybrid) cloud (which may be used to access more computing resources, and/or for check-pointing and backup). Communication infrastructure may include any suitable type of network allowing transport of data communications across thereof. The communication infrastructure may couple devices so that communications may be exchanged, such as between perceptors, servers and client devices or other types of devices, including between wireless devices coupled via a wireless network, for example. In one embodiment, the communication infrastructure may include a communication network, e.g., any local area network (LAN) or wide area network (WAN) connection, cellular network, wire-line type connections, wireless type connections, or any combination thereof.

Computing and storage infrastructure, as described herein, may be comprised of at least a special-purpose digital computing device including at least one or more central processing units and memory. The computing and storage infrastructure may also include one or more of mass storage devices, wired or wireless network interfaces, input/output interfaces, and operating systems, such as Windows Server, Mac OS X, Unix, Linux, FreeBSD, or the like. According to one embodiment, data storage infrastructure may be implemented with blockchain-like capabilities, such as none-forgeability, provenance tracking, etc. Design of the backplane 104 may also be self-contained where outside power and cloud connectors serve merely as auxiliary components.

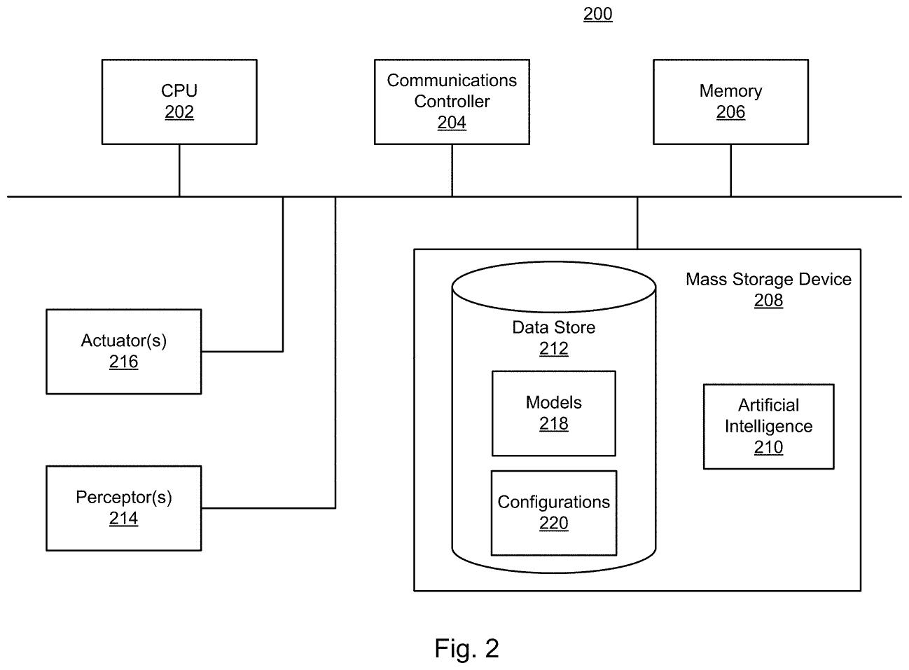

FIG. 2 presents exemplary computing and storage infrastructure according to one embodiment. A system 200 is depicted in FIG. 2 which includes a CPU (central processing unit) 202, communications controller 204, memory 206, mass storage device 208, perceptor(s) 214, and actuator(s) 216. Perceptor(s) 214 may include sensors and on-sensor silicone from the perceptor subsystem of the foreplane 102. Actuator(s) 216 may include hardware from the actuator subsystem of the foreplane 102. Mass storage device 208 includes artificial intelligence 210 and a data store 212 that contains models 218 and configurations 220.

Models 218 may be trained by providing data sets to artificial intelligence 210 to learn relationships and responses to satisfy certain goals or objectives. The learned relationships may be further calibrated with configurations 220. Configurations may include settings, preferences, policies, rules, laws, etc. Additionally, the artificial intelligence 210 may include self-containment and “smart” logic to manage resources of the physical sphere (e.g., communication, power, computing and storage). For example, perceptor(s) 214, such as cameras, may be provided in the spatial area of an enclosure. The domain of the enclosure may be configured as a home. Models 218 may recognize and track common objects associated with a home (e.g., keys, phones, bags, clothes, garbage bin, etc.). A user/customer may call upon a “memory service” and ask, “where is my key?”, “when did I take out the garbage bin?” etc.

One or more elements of the backplane 104 may be spatially aware, such as communication, computation, and storage. For example, the computations performed by the backplane may be fully spatial-aware where during the installation and configuration time, the computing system is provisioned with the absolute spatial coordinates of the enclosure it is configured for (e.g., by adopting a global positioning system (GPS) for latitude, longitude, and altitude coordinates). Each perceptor and actuator may be configured to track its relative spatial position in relation to a corresponding enclosure. The backplane 104 may create a representation of every state of the enclosure such that a representation of every physical factor, object, and actor, can have their spatial attributes accurately computed and reflected.

According to one embodiment, the communication, storage, and computation may further be spatial-tethered (with a unique spatial signature). Spatial-tethering may comprise a stronger mode of operation that can be used to configure an enclosure to operate with. Being spatially-tethered may require that all computations must be conducted by local computing resources within an enclosure. A benefit for the spatially-tethered operating mode is to ensure strict data containment and privacy such that no information will leak to any potential digital medium/devices outside of the enclosure by design.

Each device within the enclosure may be given a spatial signature. Each such device may be installed “to know” its spatial position and the device can interact with and perform operations on computation/communication payloads that originated from/destined for devices that are within the enclosure. Computation devices/nodes within an enclosure may be configured to include innate spatial-awareness. Perceptors, actuators, backplane components (e.g., network/Wi-Fi routers, computing nodes, storage nodes, etc.) may include physically built-in location beacons. One or more devices can be configured as spatial beacon masters with absolute spatial coordinates (e.g., latitude, longitude, altitude). All other devices may have relative spatial position information relative to the master(s).

Cryptographic means may be implemented to take account of spatial signatures of all the devices and computation/communication payloads to ensure that such spatial signatures cannot be tampered with. All software and computations may be programmed to be spatial aware. Each computing/storage/communication operator may only take operand (payload) that's tagged with spatial attributes known to be within the spatial confine of the physical enclosure. This way, it can be computationally guaranteed that information will not breach outside of the spatial bounds of the enclosure.

An “intelligent enclosure” by and of itself may be a computer, or a complete computing system. Any state (past state or future desired state) of the enclosure, any event that happened/can happen in and near the enclosure, and any sequence of events, is “computable.” Any state can be expressed as a sequence of computations by getting data from perceptors, updating the models and semantic space, computing steps of control, sending control signals to the actuators. Acquiring information, applying mathematical functions to process the information, and using information to affect the enclosure, can all be expressed through computation. With programming language and tools, any intelligent enclosure is programmable, to enable and achieve intended goals. In essence, enclosed space and time, with the augmentation into an intelligent enclosure, becomes computable and itself becomes a computer.

According to another embodiment, the disclosed system may be configured as an ephemeral computing system. Processed digital signals may be discarded in a fashion similar to biological systems where the eyeball/retina does not store input. Another approach is implement volatile memory in the disclosed system to ensure that there is no durable capture of sensor-captured raw information. Yet another approach may be to enable durable memory through verifiable software that performs periodic deletion. An additional approach may include the application of cryptographical mechanisms so that it will be increasing expensive/infeasible to “remember” or “recall” raw sensor data.

According to one embodiment, development and deployment of tethered computing systems may be implemented using cloud computing. A cloud service may be provided to offer a virtual-enclosure service for customers. A digital-twin of a physical enclosure may be created and operated on the cloud. A digital description and specification of the enclosure may be provided, and a virtual machine may be provisioned for each of the devices (perceptor, actuator, compute/storage/network nodes) of the enclosure. The physical backplane may be initially virtual (via the cloud virtual machine). A cloud connector may be created for the enclosure to transmit data for the relevant perceptor/actuator. Cryptographic mechanisms may be applied to encrypt all data in the cloud and access of data may require digital signatures unique to owner(s) of the enclosure.

Additionally, a marketplace may be provided to allow people to buy and own rights for a digital twin of a physical enclosure. Each digital-twin maps to its corresponding physical enclosure. People can sell and/or trade their digital-twin ownership as well as lease their digital-twin rights. An operator of the marketplace may deploy and operate enclosure services for the corresponding rights owners.

Embodiments of the present disclosure are not limited to provisioning physical enclosures into tethered computing systems. In a similar fashion, autonomous actors (e.g., cars) may also be provisioned into a self-contained computing system according to the disclosed system. Additionally, biological entities, such as animals and plants may be configured as a tethered computing system where information of the biological entities can be captured, processed, and acted upon to achieve a desired goal or maintain a predetermined state. Furthermore, computing systems may be implemented upon open environments (e.g., smart city, smart farms) or an open area (e.g., a city, a park, a collage campus, a forest, a region, a nation). All contained entities (e.g. river, highway) are observable and computable (e.g., learn, model, determine). Some of entities may be active (with perceptors and actuators) and some may be passive (observable but not interactable). To a further extent, planetary computing systems (e.g., space endeavors and inter planetary transportation, space stations, sensors (mega telescopes, etc.)) may also be established according to features of the disclosed system.

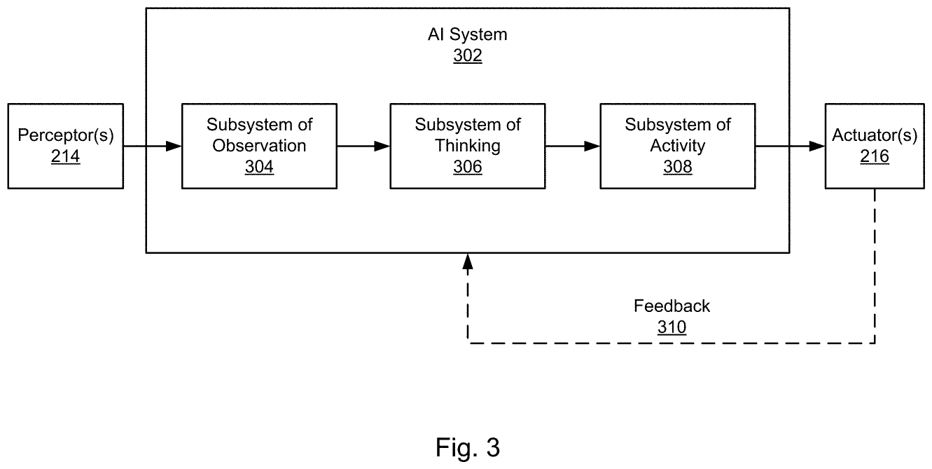

The digital sphere may comprise data and computational structures that interface with the spatial and temporal elements of the physical sphere. FIG. 3 depicts an exemplary digital sphere including AI system 302 that is coupled to elements from the physical sphere. The AI system 302 comprising a subsystem of observation 304, a subsystem of thinking 306, and a subsystem of activity 308. The AI system 302 may comprise software and algorithms configured to interoperate with each other to perform desired functions and objectives based on a given application model. The subsystems may operate under policy-based management through the administrator console. Policies may be updated or evolved through manual intervention to allow policy enablement and changes in intelligence behavior. Additionally, behaviors of the subsystems may be configured to account for laws, ethics, rules, social norms, and exceptions, that may be localized or applied universally.

The AI system may be configured with or learn rules and actions to control a plurality of devices, and/or have the devices operate automatically, for instance, according to desired spatial settings, experiences, or goals. The AI system may be trained to accommodate and assimilate spatial form factors that account for the geometry of an enclosed space via separators (e.g., wall, floor, ceiling, open-space perimeter), functional components (e.g., door, window, etc.), interior and exterior (shape, color, material: wood, brick, etc.), objects (physical entities contained within (furniture, appliance) and adjacent of the exterior), actors (e.g., biological (human, animal) or mechanical (robots, drones)), and environment (e.g., temperature, air, lighting, acoustic, utility (power, water) etc.). Temporal dimension factors including present, past, history of events, activity sequence of actors, and environment changes may also be learned and modeled by the AI system. Collectively, the spatial and temporal elements may comprise a state of the enclosure that may be monitored by the AI system. According to one exemplary embodiment, an AI system for a workplace with a set of rooms can be configured to sense and control the room temperatures in a way that is energy efficient while meeting employee needs. In this exemplary scenario, the system may observe and learn the patterns of each person. The system may change and control the temperature for various rooms and build up models that capture human patterns. With that knowledge, the system can, through the actuators, control the temperature of the rooms in a way that is most energy efficient. The same approach can be employed for achieving a variety of goals, from automating tasks to enriching human experiences.

The subsystem of observation 304 may include logic for monitoring or sensing data that can be sensed by the perceptor subsystem (e.g., perceptors 214) including structures, actors, actions, scenes, environments, etc. Each perceptor (or sensor) can be configured to perform a well-defined set of roles. For example, a camera sensor can be installed through a hole on the front door and configured to be outside-facing to watch outside activities, to recognize certain faces, and to trigger alarms as needed. Generally, sensors may cover some specific spatial areas and “sense” (or “monitor”) certain specific type of analog signals (optical, sound wave, heat, motion, etc.). The perceptor subsystem may map physical signals received by perceptors 214 into digital representations for the subsystem of observation. Parameters of observation including coverage, resolution, latency/frequency may be configured according to needs or application.

The subsystem of thinking 306 may conduct ongoing learning (e.g., memorization and generalization) and model building using data from observation system 304 to establish domain models that are representative of the data and how the data behaves and interacts with each other. In particular, the domain models may comprise specific ontological structures that support domain themes, such as a retail floor, school, hospital, legal office, trading floor, hotel, etc. As an example, the subsystem of thinking 306 for an enclosure that's used for a school may take as prior the domain knowledge of a school and the ontological structure to represent the relevant knowledge of a school. Data received from perceptors (e.g., camera array, microphone array, motion sensors, etc.) can be projected into an embedding space that is consistent with a school ontology (teacher, students, class, story-telling, etc.). Similar approaches can be applied to other themes and semantic domains. Any aspect of the enclosure may be digitally “knowable” and modeled. Objective functions (goals) of the subsystem of thinking 306 may be provisioned via the administrator console (or via artificial general intelligence).

The subsystem of activity 308 may provide “computable” and “actionable” decisions based on the modeling and learning (e.g., the state of the enclosures) of the subsystem of thinking 306 and act on these decisions via the actuator subsystem's controls (actuator(s) 216). The decisions may be related to human-spatial experiences or objective functions including a sequence of tasks to achieve certain goals defined by a modeled application. The decisions may be based on triggers in response to a recognition of object, actor, events, scenes, etc. An example may include scanning an owner's face by the observation system 304, sending the scan of the face to the subsystem of thinking 306 that has learned to recognize the owner's face, and making a decision with the subsystem of activity 308 to open a door in response to the owner's face. According to another example, the observation system 304 may detect someone trying to get up in bed; the subsystem of thinking 306 may recognize the action and make a decision with the subsystem of activity 308 to turn on the lights. Additionally, AI system 302 may receive feedback 310 through the action of the actuator(s) 216 which may include data that can be used by AI system 302 to improve its functionality and decision making.

The disclosed system may also provide a macro enclosure architecture where multiple enclosures can be composed into a compound enclosure. An enclosure that does not contain any other enclosure within itself may be referred to as an atomic enclosure. A compound enclosure may comprise an enclosure within another enclosure, such as by “unioning” a plurality of enclosures together, stacking vertically one or more enclosures on top of another, or by merging a plurality of enclosures together. This compositional macro structure enables intelligent enclosures to be installed and deployed and expanded gradually based on needs.

FIGS. 1 through 3 are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

It should be understood that various aspects of the embodiments of the present invention could be implemented in hardware, firmware, software, or combinations thereof. In such embodiments, the various components and/or steps would be implemented in hardware, firmware, and/or software to perform the functions of the present invention. That is, the same piece of hardware, firmware, or module of software could perform one or more of the illustrated blocks (e.g., components or steps). In software implementations, computer software (e.g., programs or other instructions) and/or data is stored on a machine-readable medium as part of a computer program product and is loaded into a computer system or other device or machine via a removable storage drive, hard drive, or communications interface. Computer programs (also called computer control logic or computer-readable program code) are stored in a main and/or secondary memory, and executed by one or more processors (controllers, or the like) to cause the one or more processors to perform the functions of the invention as described herein. In this document, the terms “machine readable medium,” “computer-readable medium,” “computer program medium,” and “computer usable medium” are used to generally refer to media such as a random access memory (RAM); a read only memory (ROM); a removable storage unit (e.g., a magnetic or optical disc, flash memory device, or the like); a hard disk; or the like.

Computer programs for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as C++, or the like, and procedural programming languages, such as the “C” programming language or similar programming languages.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

What is claimed is:

1. A system for providing an enclosure with intelligent computing capabilities, the system comprising:
a physical sphere including physical spatial elements and temporal elements associated with the enclosure;
a fusion system comprising:
a foreplane including physical fabric, a perceptor subsystem, and an actuator subsystem, and
a backplane including a communication infrastructure, computing and storage infrastructure, power infrastructure, redundancy, and cloud connections; and
a digital sphere including an artificial intelligence (“AI”) system coupled to the physical sphere by the fusion system, the AI system comprising:
a subsystem of observation configured to receive data from the perceptor subsystem, the data corresponding to the physical spatial elements and the temporal elements,
a subsystem of thinking configured to learn from, model, and determine a state of the enclosure based on the received data, and
a subsystem of activity configured to generate decisions with the actuator subsystem based on the state of the enclosure according to a predetermined objective for the enclosure.

2. The intelligent enclosure system of claim 1 wherein the perceptor subsystem comprises one or more devices that include one or more sensors, on-sensor computing silicon, and embedded software.

3. The intelligent enclosure system of claim 1 wherein the perceptor subsystem comprises at least one of optical, auditory, motion, heat, humidity, and smell sensors.

4. The intelligent enclosure system of claim 1 wherein the perceptor subsystem comprises at least one of phone, camera, robotic, drones, and haptic devices.

5. The intelligent enclosure system of claim 1 wherein the perceptor subsystem comprises medical equipment that assesses a state of health for biological actors within the enclosure.

6. The intelligent enclosure system of claim 1 wherein the subsystem of thinking is further configured to model the received data according to a domain theme.

7. The intelligent enclosure system of claim 6 wherein the domain theme includes at least one of a retail floor, school, hospital, legal office, trading floor, and hotel.

8. The intelligent enclosure system of claim 6 further comprises an enclosed physical space that serves a defined social economical purpose.

9. The intelligent enclosure system of claim 6 wherein the generated decisions include tasks to achieve functions according to the domain theme.

10. The intelligent enclosure system of claim 1 wherein the subsystem of thinking is further configured to build a model of the physical sphere, wherein the model includes a description of a semantic space and ongoing actions of the physical sphere.

11. The intelligent enclosure system of claim 10 wherein the AI system is configured to train the model by learning relationships and responses to satisfy given goals or objectives based on a domain theme.

12. The intelligent enclosure system of claim 11 wherein the AI system is further configured to calibrate the learned relationships based on configurations including at least one of settings, preferences, policies, rules, and laws.

13. The intelligent enclosure system of claim 10 wherein the subsystem of thinking is further configured to use domain-specific deep-learning algorithms and overall life-long learning to improve the model.

14. The intelligent enclosure system of claim 1 wherein the state of the enclosure comprises a combination of the physical spatial elements and the temporal elements that is monitored by the AI system.

15. The intelligent enclosure system of claim 1 wherein the backplane is spatial-aware and the communication infrastructure, computing and storage infrastructure, power infrastructure, redundancy, and cloud connections of the backplane are tagged with spatial-signatures that prohibit tampering.

16. The intelligent enclosure system of claim 15 wherein the backplane performs computation operations that ensure information is contained within the physical enclosure.

17. The intelligent enclosure system of claim 1 wherein the physical spatial elements comprise features associated with a geometry of the enclosure including separating structures, an interior and exterior of the enclosure, objects, actors, and environment.

18. The intelligent enclosure system of claim 1 wherein the temporal elements include factors related to time, events, and environmental changes.

19. The intelligent enclosure system of claim 1 wherein the subsystem of activity is further configured to use the actuator subsystem to induce changes in the physical sphere based on the generated decisions.

20. The intelligent enclosure system of claim 1 wherein the actuator subsystem comprises digital controls for equipment, appliance, mechanical, and perimeter objects.
